(19) United States
(12) Patent Application Publication
(10) Pub. No.: US 2010/0143857 A1
(43) Pub. Date: Jun. 10, 2010

(54) PHOTO-ACTIVATED DISINFECTION

(76) Inventor: Michael John Colles, Sussex (GB)

Correspondence Address: FAY SHARPE LLP, 1228 Euclid Avenue, 5th Floor, The Halle Building, Cleveland, OH, 44115, US

(21) Appl. No.: 12/443,905

(22) PCT Filed: Oct. 4, 2007
(86) PCT No.: PCT/GB2007/050612

§ 371 Date: Feb. 16, 2010

(30) Foreign Application Priority Data
Oct. 4, 2006 (GB) 0619588.7

(51) Int. Cl. A61N 5/06

(52) U.S. Cl. 433/29

(57) ABSTRACT

Photo-activated disinfection in the oral cavity is performed by a dental apparatus which includes a handpiece having proximal and distal portions, and a handpiece cover. The distal portion of the handpiece includes light emitting elements. The handpiece cover includes a sleeve adapted to provide a cover to the proximal portion of the handpiece and a sheath adapted to provide a cover to the distal portion of the handpiece. Suitably, the sleeve is a disposable component.

The present invention relates to photo-activated disinfection. In particular, the present invention relates to photo-activated disinfection in the oral cavity.

EP 637 976 describes the use of photosensitising compounds in killing microbes involved in a number of oral diseases by irradiation with laser light. The process involves gaining access to the treatment site, contacting the tissues wound or lesion with a photosensitising composition and irradiating the tissues, wound or lesion with laser light at a wavelength absorbed by the photosensitising composition.

This process is taken further in our earlier patent application, WO00/62701 in which we describe a minimally invasive process and apparatus for treating dental caries. A small tunnel is prepared from the outer surface of the tooth to the site of the carious lesion. The carious dentine is then inoculated with a light photosensitising composition, such as those described in EP 637 976. An optical fibre is inserted into the tunnel. A proximal end of the fibre is coupled to a laser-light generator. The distal tip of the fibre is shaped to spread light around substantially the whole of the tooth cavity. As the photosensitising composition, we have preferred the use of Toluidine Blue O (TBO), requiring activation by laser light at a wavelength of 635 nm and with a power of approximately 100 mW.

This process has been found to be highly effective. However, sources of suitable laser light are expensive and there is a requirement for the light to be delivered close to the treatment site.

In our earlier application, WO2004/103471 we described a dental apparatus comprising a pair of optical guides training light from an LED or LED array onto two external sides of a tooth.

The present invention relates to further innovative developments of the invention described in our earlier application.

According to the present invention, there is provided an apparatus, in particular a dental apparatus, comprising a handpiece having proximal and distal portions; and a handpiece cover. The handpiece comprises means for emitting light from the distal portion thereof.

Preferably, the handpiece cover comprises a sleeve adapted to provide a cover to the proximal portion of the handpiece and a sheath adapted to provide a cover to the distal portion of the handpiece. Suitably, the handpiece cover is formed as a disposable component or as a sterlisable material, for example, from an autoclavable material. Suitably, the handpiece cover is formed as a disposable component and the sheath includes an optical window to allow transmission of light from the handpiece. Alternatively, the sheath is made substantially completely from an optically transmissive material.

Alternatively, the handpiece cover is formed as a disposable, unitary component.

Suitably, the handpiece is adapted to emit light from a side face thereof.

Preferably, the handpiece includes a light source for generating the light for emitting from the distal portion of the handpiece. More preferably, the handpiece further comprises cooling means, adapted to cool the light source. Suitably, the cooling means is a water-cooling device or a heat pipe.

Suitably, the light source is a filtered white light source. Preferably, the light source is a light emitting diode or array of light emitting diodes. Preferably, the light has a wavelength of from 550 to 690 nm, more preferably from 600 nm to 680 nm, even more preferably from 610 to 640 nm.

Most preferably, the light source comprises an array of light emitting diodes. More preferably, the light source comprises a multiplexed arrays of light emitting diodes.

In one embodiment, the apparatus further includes a supplementary light source and respective supplementary optical transmission means; wherein the supplementary optical transmission means is shaped and dimensioned for entry into a tooth cavity.

The present invention also provides a method for disinfection of tissues, in particular tissues of the oral cavity or a wound or lesion in the oral cavity, the method comprising contacting the tissues, wound or lesion with a photosensitising composition, irradiating the tissues, wound or lesion with light at a wavelength absorbed by the photosensitising composition.

The method is characterised in that the light source is provided by a dental apparatus as described above.

Preferably, the photosensitising composition comprises at least one photosensitiser selected from toluidine blue O, methylene blue, dimethylene blue or azure blue chloride. More preferably the photo sensitiser is toluidine blue O. Most preferably, the sensitises in toluidine blue O in the form of ‘tolonium chloride’, being the pharmaceutical grade of TBO wherein the purity and isomeric ratios are maintained.

The above and other aspects of the present invention will now be described in further detail, by way of example only, with reference to the accompanying drawings, in which:

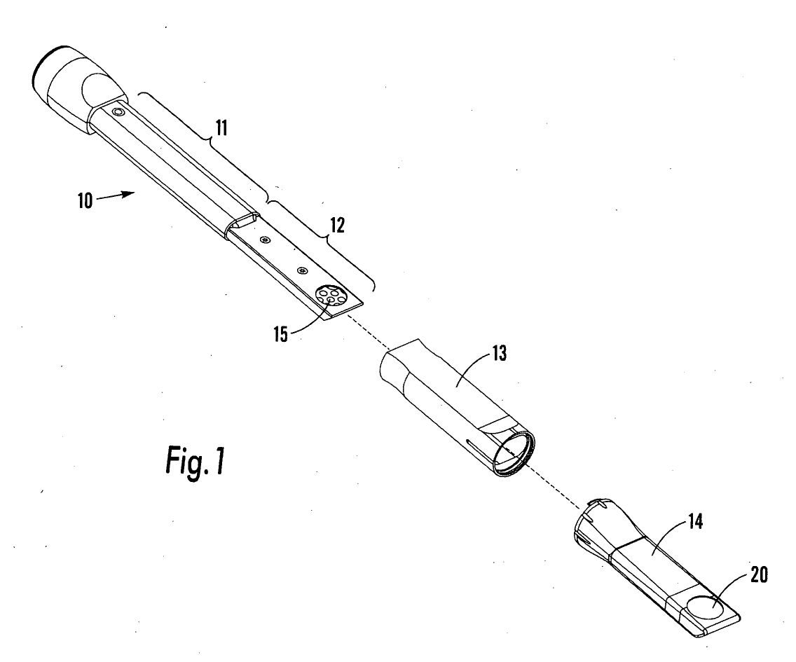

FIG. 1 is a perspective disassembled view of an embodiment of a dental apparatus in accordance with the present invention; and

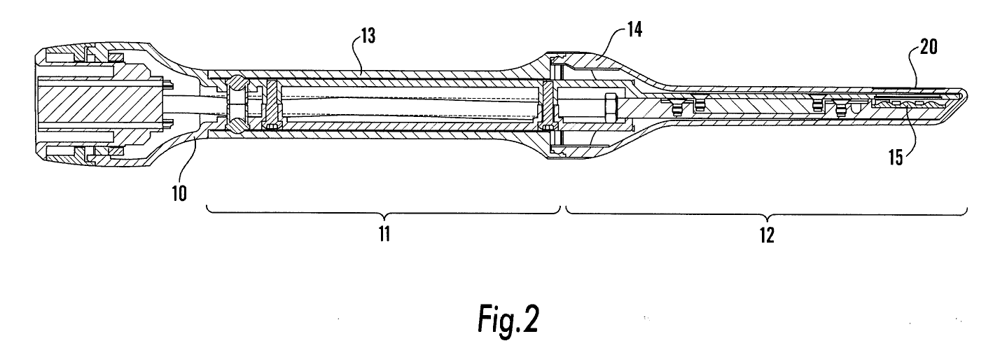

FIG. 2 is a cross-sectional view of the apparatus of FIG. 1 in an assembled configuration.

The figures show one embodiment of a dental apparatus in accordance with the present invention. The apparatus comprises a handpiece 10 having a proximal portion 11 and a distal portion 12 and a handpiece cover comprising a sleeve 13 for the proximal portion 11 of the handpiece 10 and a sheath 14 for the distal portion of the handpiece 10.

Handpiece 10 includes a light source 15 in the distal portion of the handpiece. In the preferred embodiments, the light source comprises an LED (light-emitting diode) or, more preferably, an array of LEDs. A particularly suitable light source comprises a multiplexed array of LEDs in which multiple LEDs are bundled as a cluster of LEDs and a plurality of clusters are arranged as an array.

Sleeve 13 is adapted to provide a cover for the proximal portion 11 of the handpiece 10, which is the portion which, in use, will be held by the dental surgeon. The sleeve will, accordingly, typically be made from a hard anodised autoclavable material. However, the sleeve 13 may, alternatively, be manufactured from a disposable material such as a plastics material.

Sheath 14 provides a cover for the distal portion of the handpiece 10, which is the portion from which the light is, in use, emitted. Sheath 14 is designed as a disposable component to eliminate cross-contamination between patients. The sheath may be manufactured from a wholly optically-transparent material (in the sense of being optically transparent at the desired wavelengths) or from a non-transparent material, in which case the sheath 14 is provided with an optically transparent or inert window 20.

In an alternative embodiment (not shown), sleeve 13 and sheath 14 are formed as a unitary component, providing a cover for both the proximal and distal portions of the handpiece. Such a unitary component is most suitably manufactured as a wholly disposable element, including, as described above, an optical window 20 as required.

The handpiece 10 is operatively coupled to a base unit (not shown) which provides an electrical supply to the light source at the required voltage and current. Typically, the handpiece is detachable from the base unit. Those skilled in the art will be readily able to devise suitable electrical coupling arrangements.

Additionally, the handpiece 10 houses such cooling devices as may be necessary to maintain either the handpiece generally or the light source at the correct operating temperature. For example, in preferred embodiments, the handpiece 10 includes a water circulating system or circuit and the handpiece 10 includes means for operatively coupling the handpiece to a base unit which includes a water-cooling apparatus.

In alternative embodiments, a heat pipe provides the necessary cooling.

In this invention we have sought to use light emitting diodes (LEDs) because they are available as compact low-cost sources. As described above, they are available as multiplexed arrays, typically comprising 600 or more individual LEDs, with substantial output powers. The output wavelength spread of such devices, whilst not nearly as narrow as that of a laser, are still substantially narrower than any other parameter of significance such as the absorption profile of Toluidine Blue O (TBO) or variations in dental hard and soft tissue transmission. For example, a particularly suitable LED array is that supplied by Lamina Ceramics under their trade name BL-2000 series. That array provides an output in a broad band at around 618 nm. This coincides with the shoulders of the activation peak of tolonium chloride, which centres on about 620 nm. The band is less precise than that achievable with a laser diode, but this reduced effectiveness is more than adequately counterbalanced by power outputs of the order of 700 mW or more (some seven or more times the output of a laser diode).

The concentration of tolonium chloride will depend on the specific application and chosen wavelength. We have determined an optimum for caries of 12 μg/ml. Longer wavelengths and other applications especially periodontal disease where in vivo dilution may occur are likely to need greater concentrations. Preferably the concentration should fall within an overall range between 10 μg/m1 to 1 mg/ml.

To illustrate the invention, in vitro trials were conducted against Strep Mutans NCTC 10499 and Porphyromonas gingivalis, bacteria associated with caries and periodontitis. The apparatus selected used a Lamina Ceramics BL 2000 LED array, approximately 16-18 mm in diameter, operating at 640-660 nm, in combination with aqueous tolonium chloride (25.4 mg/l) as the photosensitiser.

Evaluation was carried out on bacteria in planktonic suspensions in opaque-walled flat-bottomed sample vials (16-18 mm diameter to correspond with the dimensions of the LED array). Bacterial broth (80 ml) was mixed with an aqueous solution of the photosensitiser (80 ml), or sterile saline (80 ml) for the control samples, giving a total volume of 160 ml. The concentration of bacteria in the final mixed solutions was in the range 2.4×10⁷ to 1.6×10⁹ cfu/ml and the final concentration of tolonium chloride was 12.7 mg/l.

The LED array of the apparatus was positioned directly above the liquid at the top of the sample well, spaced 20 mm therefrom, such that all the light from the array was directed into the well. Each sample was irradiated with a known energy dose. Serial dilutions of the suspension were made followed by plating out to determine the number of viable bacteria which survived the effects of either light alone (indicated as [L+S−] in Table 1 below), being the control set, or light and photosensitizer (indicated as [L+S+]), being the experimental set). Bacterial kills were calculated in terms of logarithmic reductions of bacteria from the initial value. The range of results obtained, the mean reduction and the standard denation σ is given in the table.

TABLE 1

| | | [L+S−] | | | [L+S+] | | |
|---|---|---|---|---|---|---|---|
| | Power | Range | | | Range | | |
| Total Energy | (mW) × | of log | Mean log | | of log | Mean log | |
| (J) | Time (s) | reduction | reduction | σ | reduction | reduction | σ |

Streptococcus mutans

| | | | | | | | |
|---|---|---|---|---|---|---|---|
| 4.1 | 138 × 30 | 0-0.9 | 0.38 | 0.39 | 2.7-4.8 | 3.85 | 1.0 |
| 8.3 | 138 × 60 | 0-0.4 | 0.27 | 0.15 | 2.8-4.3 | 3.99 | 1.25 |
| 17.0 | 283 × 60 | 0.2-0.6 | 0.34 | 0.16 | 4.6-8.1 | 6.31 | 1.41 |

Porphyromonas gingivalis

| | | | | | | | |
|---|---|---|---|---|---|---|---|
| 4.1 | 138 × 30 | 0-0.04 | 0.01 | 0.02 | 7.2-9.2 | 8.6 | 0.85 |
| 8.3 | 138 × 60 | 0-0.2 | 0.06 | 0.10 | 8.5-9.2 | 8.98 | 0.30 |
| 17.0 | 283 × 60 | 0-0.1 | 0.35 | 0.43 | 8.5-9.2 | 8.97 | 0.31 |

Light alone [L+S−] produced very small kill levels, the highest single sample reduction being a log reduction of just 1. A combination of light and photosensitizer killed significantly higher numbers. For example, using 17 J energy showed a 6.3 log reduction against S. mutans and a 9.0 log reduction against P. gingivalis. In context, whereas an initial bacteria concentration of 10⁹ cfu/ml would be reduced to not less than 10⁸ cfu/ml by light alone only 500 cfu/ml of S. mutans would survive and only one 1 cfu/ml of P. gingivalis.

1. An optical apparatus comprising a handpiece having proximal and distal portions; and a handpiece cover; wherein the distal portion of the handpiece comprises light emitting means and wherein the handpiece cover comprises a sleeve adapted to provide a cover to the proximal portion of the handpiece and a sheath adapted to provide a cover to the distal portion of the handpiece.

2. An optical apparatus as claimed in claim 1 wherein the sleeve is formed as a disposable component.

3. An optical apparatus as claimed in claim 1 wherein the sleeve is formed from a sterilisable material, preferably an autoclavable material.

4. An optical apparatus as claimed in claim 1 wherein the sheath comprises an optical window.

5. An optical apparatus as claimed in claim 1 wherein the sheath is formed substantially completely from an optically transmissive material.

6. An optical apparatus as claimed in claim 1 wherein the sleeve and sheath are formed as a unitary element.

7. An optical apparatus as claimed in claim 1 further comprising a light source for generating light for emission from the distal portion of the handpiece.

8. An optical apparatus as claimed in claim 7 wherein the light source is formed integrally with the handpiece.

9. An optical apparatus as claimed in claim 8 further comprising cooling means adapted to cool the light source.

10. An optical apparatus as claimed in claim 9 wherein the cooling means is a watercooling device or a heat pipe.

11. An optical apparatus as claimed in claim 7 wherein the light source is a filtered white light light source.

12. An optical apparatus as claimed in claim 11 wherein the light source is a light emitting diode or array of light emitting diodes.

13. An optical apparatus as claimed in claim 12 wherein the light source is a multiplexed array of light emitting diodes.

14. An optical apparatus as claimed in claim 7 wherein the light source emits light having a wavelength of from 550 to 690 nm, preferably 590 to 650, more preferably 600 to 640 nm.

15. An optical apparatus as claimed in claim 1 further comprising supplementary optical transmission means, wherein the supplementary optical transmission means is shaped and dimensioned for entry into a tooth cavity.

16. An optical apparatus as claimed in claim 15 further comprising a supplementary light source associated with the supplementary optical transmission means.

17. An optical apparatus as claimed in claim 1 further comprising a photosensitising composition.

18. An optical apparatus as claimed in claim 17 wherein the photosensitising composition comprises at last one photosensitiser selected from toluidine blue O, methylene blue, dimethylene blue or azure blue chloride.

19. A dental apparatus comprising an optical apparatus as claimed in claim 1.

20. A disinfection apparatus comprising an optical apparatus as claimed in claim 1.

21. An apparatus for disinfection of tissues in a human or animal body, the apparatus comprising an optical apparatus as claimed in claim 1.

22. A method for disinfection of tissues in a human or animal body, the method comprising the steps of i) contacting the tissues with a photosensitising composition; and ii) irradiating the tissues with light at a wavelength absorbed by the photosensitising composition; characterised in that the light is provided by a dental apparatus as claimed in claim 1.

23. A method as claimed in claim 22 wherein the tissues are tissues of the oral cavity or a wound or lesion in the oral cavity.
